(19) United States
(12) Patent Application Publication
(10) Pub. No.: US 2025/0221769 A1
(43) Pub. Date: Jul. 10, 2025

(54) System and method for combined thermal and photodynamic therapy of malignant tumors

(71) Applicant: SpectraCure AB, Lund (SE)

(72) Inventors: Sune Svanberg, Lund (SE); Katarina Svanberg, Lund (SE)

(21) Appl. No.: 18/855,911

(22) PCT Filed: Apr. 11, 2023
(86) PCT No.: PCT/EP2023/059461

§ 371 Date: Oct. 10, 2024

Related U.S. Application Data
(60) Provisional application No. 63/329,499, filed on Apr. 11, 2022.

(51) Int. Cl. A61B 18/20; A61B 18/00; A61N 5/06

(52) U.S. Cl. CPC A61B 18/20; A61N 5/062; A61B 2018/00589; A61B 2018/00642; A61B 2018/00666; A61B 2018/00791; A61B 2018/00904; A61B 2018/2005; A61N 2005/0628; A61N 2005/063

(57) ABSTRACT

A system and method for treatment of malignant tumors where a combination of a laser-light thermal therapy and a light-activated photodynamic therapy is conducted wherein regions close to emitting fibers being eradicated by thermal effects, while regions further away being treated with the light-activated photodynamic therapy and tumorcell selectivity.

### BACKGROUND OF THE INVENTION

### Field of the Invention

This disclosure pertains to applying a combination of interstitial thermal and photodynamic therapy (PDT) for eradicating malignant tumors. A region close to the fiber will be eliminated by heat/coagulation, while laser radiation will penetrate further into the tissue with reasonable intensity, which will now cause selective tumor cell death in more distal regions. Thus, tumor cells will be eradicated in an extended volume while sparing tissues at risk in the peripheral regions, where only selective, non-thermal PDT action is utilized.

### Description of the Prior Art

Malignant tumors can be eradicated by photodynamic therapy (PDT), which is based on a combination of an administered photosensitizing drug, a sufficient level of activating light of proper wavelength, normally derived from a laser, and the availability of oxygen in the tissue. In PDT with sensitizers such as Photofrin, Metvix, and Foscan, the treatment action is due to the release of tissue-toxic radicals in tumor cells. Other sensitizers, such as Tookad and Verteporfin, action on the vessels, which shut down under light irradiation and deprive the tumor from nutrition. PDT is widely applied for different types of malignant lesions. For the case of deep-lying tumors, interstitial PDT (IPDT) can be applied, where a number of optical fibers, carrying the treatment light, are inserted into the tumor mass. Light penetration in human tissue is governed by absorption and light scattering processes, which are wavelength dependent. Effective dose delivery depth without tissue heating goes from a fraction of one mm approaching up to 10 mm for wavelengths ranging from green to the near infrared region. The intensity of the light is normally restricted to a level, where PDT is efficient, but where tissue heating is limited to 2° C., to avoid thermal tissue effects.

In a special implementation of IPDT, arrangements for the combined use of all fibers for delivering therapeutic radiation and the measurement of important dosimetry parameters, such as light flux, oxygenation and sensitizer concentrations are made, as described in WO2004/100789 A1, and WO2021/240023. In dosimetry programs, for example as described in WO 2008/020050, or by K. Komolibus et al., Perspectives on interstitial photodynamic therapy for malignant tumors. J. Biomed. Opt. 26: 070604, 2021. doi: 10.1117/1.JBO.26.7.070604, optimum fiber placements are calculated and optimum light delivery is determined. The goal is to achieve full treatment of the lesion while sparing organs at risk. The consideration of the organs at risk may lead to the treatment being unnecessarily conservative with regard to the delivered dose and dose distribution, which can lead to under-treatment of the malignancies and subsequent recurrence events.

Tumors can also be eradicated by laser thermal therapy, which can also be applied in an interstitial delivery mode, as described, e.g., by D. R. Wyman, W. M. Whelan and B. C. Wilson, “Interstitial laser photocoagulation: Nd:YAG 1064 nm optical fiber source compared to point heat source,” Lasers Surg. Med. 12: 659-664, 1992, and by D. R. Wyman and W. M. Whelan, “Basic optothermal diffusion theory for interstitial laser photocoagulation,” Med. Phys. 21: 1651-1656, 1994. On heating above 60° C., tissue coagulation occurs. In contrast to PDT, the thermal therapy has no specificity for malignant cells, which is largely also the case for PDT targeting the vessels. Light flux and temperature measurements can be accomplished through the same fibers as those delivering the heating radiation. Synergy between PDT and thermal treatment (at moderate temperature increase—the hyper-thermal regime) has been reported, as described, e.g. by T. S. Mang, “Combination studies of hyperthermia induced by the Neodymium:Yttrium-Aluminum-garnet (Nd:YAG) laser as an adjuvant to photodynamic therapy,” Lasers Surg. Med. 10:173-178, 1990, and S. M. Waldow, B. W. Henderson and T. J. Dougherty, “Hyperthermic potentiation of photodynamic therapy employing Photofrin I and II. Comparison of results using three animal tumor models,” Lasers Surg. Med. 7: 12-22, 1987.

Delta-amino levulinic acid (ALA) has many attractive features as a precursor in the systemic generation of the photodynamically active substance Protoporphyrin IX. It is a readily available substance, which also occurs naturally in the human body as part of the haem cycle. It exhibits a high tumor cell specificity, can be applied systemically, orally or topically, and has a fast clearing from the human body, leading to almost negligible sunlight sensitization. ALA-mediated PDT for thin skin tumors has been described e.g. by K. Svanberg et al., “Photodynamic Therapy of Non-Melanoma Malignant Tumours of the Skin Utilizing Topical δ-Amino Levulinic Acid Sensitization and Laser Irradiation”, British J. of Dermatology 130: 743, 1994. ALA formulations such as Metvix are available from the Pharmaceutical industry and has been approved for certain indications. The only disadvantage by using ALA-type agents, is a short activation wavelength around 630 nm, leading to increased tissue absorption and scattering compared to, e.g., the case of Verteporfin (689 nm) or Tokaad (753 nm), and a resulting small PDT treatment volume.

The limited light penetration through human tissue means that an unrealistically large number of interstitial fibers would be needed for ALA-type sensitizers to successfully treat a tumor with a certain volume using interstitial PDT (IPDT). While PDT is a non-thermal treatment modality, pure thermal treatment, e.g., induced by sufficiently high laser radiation, which is absorbed in the tissue, has no cancer cell specificity.

Hence, it would be an advantage to extend the volume where tumor cells will be eradicated but at the same time keep the cancer cell specificity.

### SUMMARY OF THE DISCLOSURE

Accordingly, embodiments of the present disclosure preferably seek to mitigate, alleviate or eliminate one or more deficiencies, disadvantages or issues in the art, such as the above-identified, singly or in any combination by providing a system or method according to the appended patent claims for treatment of malignant tumors using a combination of a laser-light thermal therapy and a light-activated photodynamic therapy.

According to a first aspect of the disclosure, a system for treatment of malignant tumors is described. The system may include at least two optical members with a distal portion configured to be interstitially inserted into tissue, the at least two optical members are configured for emitting and collecting light. The system may also include a light source and a detector connected to the optical fibers at a proximal end. The system may further include a control unit configured for controlling the at least two optical members so that light is transmitted to the tissue from at least one optical member and light is detected from the tissue by at least one optical member collecting light, wherein the control unit is further configured to control a power of the light source so that a combination of a laser-light thermal therapy and a light-activated photodynamic therapy is conducted wherein regions close to emitting fibers being eradicated by thermal effects, while regions further away being treated with the light-activated photodynamic therapy and tumor-cell selectivity.

In some example of the system may the laser source be emitting a single laser wavelength used for both the laser-light thermal therapy and the light-activated photodynamic therapy.

In some example of the system may the laser source be adapted to emitting a wavelength matching requirements for a sensitizer being Protoporhyrin IX generated from the precursor δ-aminolevulinic acid (ALA), or an ALA-related formulation, used for the light-activated photodynamic therapy.

In some examples of the system may the system be configured for diagnostics which is based on at least one of the parameters: light flow between fibers, oxygenation in the tissue, concentration of sensitizer in the tissue, blood flow through the tissue, temperature at the individual fiber tips, and/or temperature dynamics.

In some examples of the system may the parameters be derived from light detected by the collecting fibres.

In some examples of the system may the control unit be configured to use measured optical data from the colleting fibers as input for treatment planning and interactive follow-up handling aspects related to the laser-light thermal therapy separately, and aspects related to the light-activated photodynamic therapy separately, using relevant models for tissue heating, heat flow, and PDT action.

In some examples of the system may at least the light flow between fibers, the temperatures at fibers, and the temperature dynamics be measured as input in the combined PDT and thermal dosimetry program, to ensure that the respective threshold doses have been delivered, and organs at risk have doses well under thresholds.

In some examples of the system may at least the light flow between fibers, the temperatures at fibers, and the temperature dynamics be expressed in time constants, or other time-dependent parameters.

In some examples of the system may the control unit be configured to extract beat signals in the kHz frequency range caused by Doppler shifts in light interaction with flowing blood cells.

In some examples of the system may each emitting fiber be connected to an individual light source having an output power in the range 0.1-5 W.

In some examples of the system may the laser source output the light at a wavelength in the range of 625-640 nm.

In another aspect of the disclosure is a method of treatment of malignant tumors described. The method may include inserting at least two optical members with a distal portion configured to be interstitially inserted into tissue, the at least two optical members are configured for emitting and collecting light and are connected to a light source and a detector at a proximal end. The method may also include controlling the at least two optical members so that light is transmitted to the tissue from at least one optical member and light is detected from the tissue by at least one optical member collecting light, and controlling a power of the light source so that a combination of a laser-light thermal therapy and a light-activated photodynamic therapy is conducted wherein regions close to emitting fibers being eradicated by thermal effects, while regions further away being treated with the light-activated photodynamic therapy and tumor-cell selectivity.

In a further aspect of the disclosure is a computer program described. The computer program may be executed on a control unit of the system according to the disclosure for carrying out the steps of:
controlling at least two optical members so that light is transmitted to the tissue from at least one optical member and light is detected from the tissue by at least one optical member collecting light,
controlling a power of the light source so that a combination of a laser-light thermal therapy and a light-activated photodynamic therapy is conducted wherein regions close to emitting fibers being eradicated by thermal effects, while regions further away being treated with the light-activated photodynamic therapy and tumor-cell selectivity.

### BRIEF DESCRIPTION OF THE DRAWINGS

These and other aspects, features and advantages of which examples of the disclosure are capable of will be apparent and elucidated from the following description of examples of the present disclosure, reference being made to the accompanying drawings, in which:

FIG. 1 is illustrating a schematic example of a treatment according to the disclosure;

FIG. 2 is illustrating schematic examples of a system to be used for a treatment of a tumour according to the disclosure;

FIG. 3 is illustrating schematic examples of a device which may be used as part of the system of FIG. 3;

FIG. 4 is illustrating a modular system based on the device in FIG. 3;

FIG. 5 is illustrating a schematic diagram of temporal development of temperature under intermittent laser irradiation; and

FIG. 6 is illustrating a calculation over light penetration and increase in light dose.

### DESCRIPTION OF EXAMPLES

Specific examples of the disclosure will now be described with reference to the accompanying drawings. This disclosure may, however, be embodied in many different forms and should not be construed as limited to the examples set forth herein; rather, these examples are provided so that this disclosure will be thorough and complete, and will fully convey the scope of the disclosure to those skilled in the art.

The following disclosure focuses on examples of applying a combination of interstitial thermal and photodynamic therapy (PDT) for eradicating malignant tumors. For example, using the readily available precursor δ-amino levulinic acid (ALA), which in the body is transformed into the photodynamically active sensitizer Protoporphyrin IX. In other examples, other sensitizers readily known to the person skilled in the art may be used. The sensitizer is selectively generated in malignant tumor tissue with high specificity and is very attractive for PDT, but the excitation wavelength around 630 nm only allows a quite limited light penetration through human tissue. Thus, an unrealistically large number of interstitial fibers would be needed to successfully treat a tumor with a certain volume using interstitial PDT (IPDT). For other sensitizers other wavelengths associated with the selected sensitizer may be used.

While PDT is a non-thermal treatment modality, pure thermal treatment, such as induced by sufficiently high laser radiation, which is absorbed in the tissue, is a further possibility, albeit with no cancer cell specificity. It is now put forward, that it is possible to increase the treated tumor volume in IPDT, such as ALA-mediated IPDT, by substantially increasing the laser power in the interstitial fibers, typically by a factor of 10 above customary levels. The regions close to the fiber will then be eliminated by heat/coagulation, while laser radiation will penetrate further into the tissue with reasonable intensity, and will now cause selective tumor cell death in more distal regions. Thus, tumor cells will be eradicated in an extended volume while sparing tissues at risk in the peripheral regions, where only selective, non-thermal PDT action is utilized. This may for example be an advantage when treating prostate cancer.

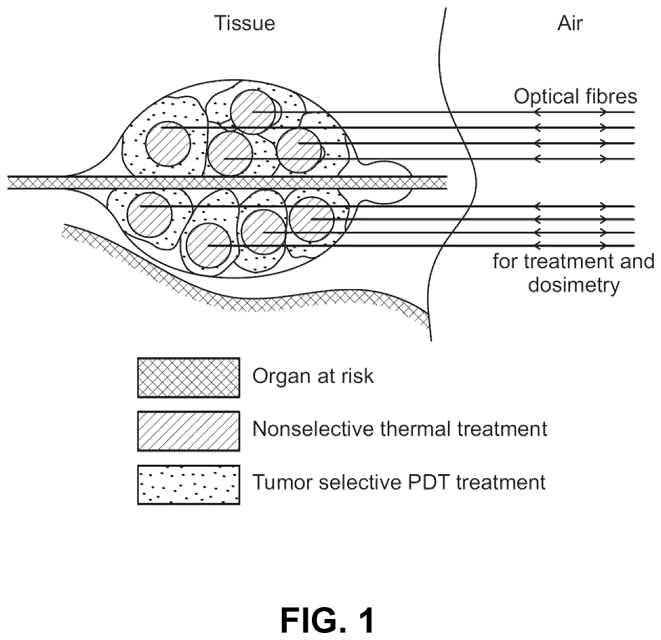

FIG. 1 is illustrating a schematic example of a treatment according to the disclosure. The treatment includes a combination of thermal and photodynamic therapy, both pursued in the interstitial treatment domain. The invention is schematically illustrated in FIG. 1, where the non-excluding application to prostate cancer is taken as an example. Other cancer types, where the present invention is equally applicable include breast cancer, brain, lung, liver and renal cancer, as well as solid tumors at other locations. The main idea of the procedure disclosed is to compensate for the limited light penetration of the light activating a sensitizer needing a low wavelength, such as Protoporphyrin-IX (PpIX) at 630-635 nm, by increasing its power, beyond the normal level, where PDT only is pursued without any accompanied heat generation. ALA-PpIX is here taken as a preferred, but a non-restraining example of application of the proposed combined treatment. The fibers are placed in the area to be treated, with sufficient distance to organs at risk for avoiding unwanted thermal effects. The elevated 630 nm laser powers would then cause heat effects in the fiber tips surroundings, i.e., in areas of malignant tissue, which at any rate should be eradicated, and where thermal and effective PDT action would in principle have similar and very efficient action. However, beyond the heated area, 630 nm light of useful intensity (because of the initially higher light level administration) would penetrate to selectively treat tumor cells, which are close to the organs at risk. A calculation with realistic parameters suggests that useful selective PDT action can be extended by 3-5 mm beyond what is possible with classical, non-thermal ALA-PDT, see FIG. 6 where the vertical axis is log(fluence rate) and the horizontal is distance in mm. This would be in strong contrast to approaches, where thermal treatment only is employed, and the non-selectivity would pose a hazard to organs at risk (such as the rectum and the urethra in the case of prostate-cancer treatment) if close-lying tumor areas should be eradicated. The same is true for other tumor-selective photosensitizers.

The approach proposed benefits from the now readily available high-power CW lasers (Watt level) at reasonable price. Further, ALA is safe and approved for many indications, and its use as an orphan drug for a new indication would be a natural step.

The approach, which we have illustrated for the case of ALA-induced Protoporphyrin IX can clearly be applied for any other suitable tumor-targeting PDT drug. Such drugs, which might be activated at longer wavelengths, and thus having better tissue penetration ability, would again use the tumor-selective action. However, when allowing the region close to the delivery fibers to operate in the thermal treatment regime, fewer fibers would be needed, and thus the procedure would be more minimally invasive. The same laser wavelength for thermal and PDT action is preferred, but different wavelengths could also be contemplated for convenience.

When operating in the combined thermal and PDT regimes, the same fibers are used for administering treatment light and for assessing light flux through the tissue, as well as sensitizer concentration (by fluorescence) and oxygenation level (using the difference in absorption spectrum for oxy- and deoxyhemoglobin) regarding PDT optimization. Likewise, temperature can be assessed by exciting rare-earth salts (thermo-phosphors) doped into the fiber tips, and emitting temperature-dependent fluorescence on laser excitation through the fiber. Also, the stop of blood flow can be assessed by laser-Doppler monitoring, detecting the beat frequency (in the kHz range) between light scattering from moving blood cells and fixed tissue structures, and can readily be integrated in a multi-fibre IPDT system, as described in a patent applications WO 03/041575 A1, WO 2004/100789 A1, WO 2008/020050 A1 and WO 2021/240023 all herein incorporated by reference. Laser-Doppler instruments are available from multiple vendors. The basic techniques are described, e.g. by P. A. Oberg, “Laser-Doppler flowmetry,” Crit. Rev. Biomed. Eng. 18:125-163, 1990.

Because of the presence of two tissue-destructive processes simultaneously in action, special care is needed in placing the interstitial fibers into the tumor mass. The thermal effect with temperature rise will non-selectively kill malignant and normal cells alike, and clearly it is important that no fiber ends up too close to any organ at risk to cause thermal damage. In the dosimetry considerations and calculations, the most important aspect must now be the temperature spatial distribution. Since in the present invention, the sensitizer agent (ALA, etc.) is highly tumor specific (while commonly used sensitizers in IPDT, such as Tookad or Verteporfin, are not), little risk for damage to non-infiltrated organs at risk pertains, and a higher light dose for PDT can be applied without damage risk.

The selective PDT process depends on the simultaneous presence of sufficient amounts of light, sensitizer and oxygenated blood, which all can be assessed optically, and quantified using computer programs.

In an example, this may be applied in a computer-based virtual planning environment. In the virtual planning environment, the positioning of light sources, such as optical fiber ends may be made. In an example, a method is used where a model for light propagation is applied. An embodiment of determining the number and position of light sources is based on such a model. A model that may be used is the transport equation for radiative transfer, and more specifically, an approximation based on the assumption of diffuse light propagation—the diffusion equation.

The task of finding the optimal fiber positions may be formulated as maximizing the light fluence rate within the target organ, while minimizing the light distribution within the organs at risk (OAR) adjacent to the target organ to be treated. The optimization algorithm may be an iterative random-search algorithm similar to a simulated annealing type algorithm.

Based on the virtual planning, the light sources may accordingly be positioned in the region of treatment tissue. This positioning may be provided by using a surgical template based on data that is output from said virtual planning.

In an example, a method is used where the decay of the transmitted light at increasing distances in the tissue is recorded and fitted to a model for light propagation. The resulting data is the effective attenuation coefficient of the tissue, evaluated using the diffusion equation. By using the effective attenuation coefficient and the diffusion equation, the fluence rate may thus be calculated in the tissue.

Information on the geometry, the actual fiber positions, and the values of the effective attenuation coefficient in the tissue may be used as input for a Cimmino optimization algorithm to predict required irradiation times for all source fibers. Furthermore, via tissue importance weighting within the Cimmino algorithm the possibility to discriminate between target tissue and organs at risk (OAR) in terms of the deposited light dose may be provided.

This system named IDOSE is further described in patent application WO 2008/020050, herein incorporated by reference. Since the optical properties of tissue can be expected to vary considerably between patients and will change when coagulation occurs, reliable input data for the PDT dosimetry (pertaining to peripheral regions, close to organs at risk, and using procedures disclosed in WO 2008/020050 must be measured for the specific patient before any appreciable thermal heating has occurred. Such data form base values for successful PDT action.

Tissue temperature rise due to laser irradiation is governed by the absorption coefficient of the tissue and the applied laser power, and clearly is strongly influenced by tissue specific heat capacity, tissue heat conductance, and heat transport by blood vessels (convection). The processes may be modelled and the resulting temperature distribution can be calculated using published procedures, such as those provided by e.g. A. L. McKenzie, “Physics of thermal processes in laser-tissue interaction,” Phys. Med. Biol. 35: 1175-1209, 1990, M. M. Chen and K. R. Holmes, “Micro-vascular contributions in tissue heat transfer,” Ann. N.Y. Acad. Sci. 335:137-150, 1980, and F. P. Incropera and D. P. De Witt, Fundamentals of heat and mass transfer, 3rd edn., (New York: Wiley, 1990), all incorporated by reference. Basically differential equations with boundary conditions are solved. Complex geometries can more accurately be handled by Monte-Carlo simulations, which can be performed at high speed using modern computers and graphical cards. The heat distribution due to light absorption can then be calculated, basically as an extension of models first developed for light-dose assessments; see e.g. L. H. Wang, S. L. Jacques, and L. Q. Zheng, “MCML-Monte Carlo modeling of light transport in multi-layered tissues”, Comp. Meth. Programs Biomed. 47: 131-146, 1995, herein incorporated by reference. The Monte-Carlo approach is in strong contrast to conventional transport theory, which is mathematically expressed by the radiative transport equation.

Clearly, large variations can be present between different patients, and thus reliable measurements on temperature rise and heat conduction dynamics/blood flow are needed for an optimum result. The temperature can be directly measured only at the fiber tips, which may be done by analyzing the spectral emission from temperature-sensitive phosphor agents, integrated into the optical fiber tips, as described in WO 03/041575 A1 which is herein incorporated by reference). Spectral distribution is assessed by a spectrometer or other spectrally sensitive device, and alternatively the light temporal decay time following pulsed or modulated light excitation can be recorded. However, the action by the irradiation can be assessed not only at the fiber tips but also inside the tissue under treatment using the laser Doppler signal in light transmitted by one fiber and recorded by other fibers, as discussed above. On coagulation, blood flow is quenched and the Doppler beat signal is reduced. Simple electronics can be used to pick up the Doppler beats in the light successively measured when passing between transmitting and receiving fiber pairs in the procedure of assessing light flow through the tissue.

The present description also discloses a further, and very effective way to assess the progression of the thermal effects/coagulation of the tissue by not only noting the instantaneous temperature value, but also focusing on the dynamics of temperature, which is strongly influenced by blood flow.

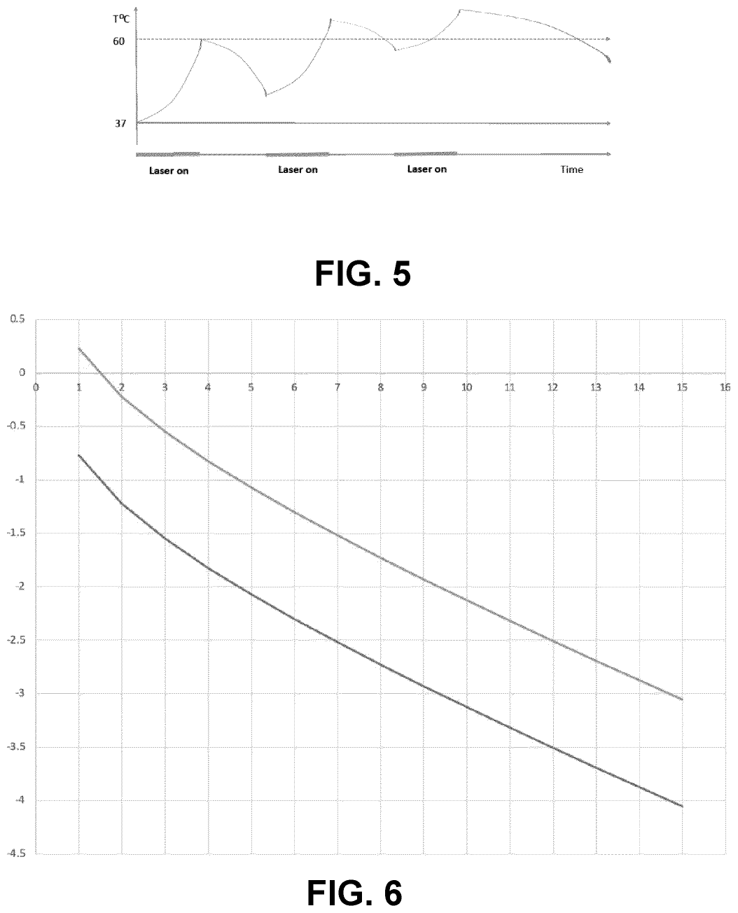

The action of heating laser radiation can be assessed using temperature information obtained at each of the fiber locations. Static data can be used as input in a thermal model, describing the progress of tissue eradication due to coagulation, as described above. However, the dynamic evolution of the temperature is not incorporated, which in contrast is an important aspect of the present disclosure. The methodology disclosed is now discussed with reference to FIG. 5.

FIG. 5 illustrates a schematic diagram of temporal development of temperature under intermittent laser irradiation. Temperatures are evaluated, but in addition, and very importantly, the time constants for the temperature development change, reflecting the development of coagulation. Three heating sessions are indicated. A plurality of such sessions of suitable duration, interlaced with cooling sessions of suitable duration may be used in practice. The curve does not intend to accurately reflect the details of temperature change, but just to illustrate heating when laser is on and cooling when laser is off, and that the dynamics change as heat transport through flowing blood is altered by coagulation.

The temperature T(t) at a particular point in tissue can for an individual, sufficiently short, light on/off period be approximatively modelled according to a mathematical expression, containing, e.g., exponential functions

e^{±t/τ}^{ᵢ}

where τᵢ is the time constant for dynamic change, pertaining to irradiation or cooling periods i. These parameters attain new values in successive heating and cooling periods, which reflects the development of coagulation with strong influence on the cooling due to the blood flow. The flow is decreasing due to the increasing coagulation in the affected regions. Typical values for the constants can be assessed in test measurements on realistic tissues, where coagulation can be followed, e.g., by MRI or by histo-pathological investigations of the irradiated tissues. The time constants τᵢ contain the dynamic information, which is specific to the present disclosure. Other mathematical functions may pertain, but always containing coagulation-dependent parameters describing the rate of change. The temperature can be measured intermittently, e.g., every 10 second. Either the relative intensity of spectral components is measured, or the emission decay time, typically in the □s time region, is evaluated. Both features are temperature dependent for thermo-phosphor compounds. Fluorescence decay times would be measured following short-pulse radiation, or by assessing demodulation and phase shift following amplitude-modulated CW laser irradiation.

As treatment progresses, heat and PDT actions are indirectly monitored optically/spectroscopically based on light flow, sensitizer concentration, degree of blood oxygenation, and temperature at the fiber tips, and by assessment of the coagulation status/propagation by Doppler flow measurements and monitoring the dynamic temperature evolution. The two latter aspects probe indirectly the status of tissue distant from the fiber tips. Tomographic routines can be applied as now performed, e.g., in the approach with regard to light flow described in WO 2008/020050 A1 and WO 2008/152076 A2, both herein incorporated by reference. All such measured parameters, which all are retrieved using the same fibers as those employed for delivering the treatment light, are fed into a dosimetry/treatment program, which basically is an extension of the presently available IDOSE program, with the heat deposition and transfer processes determined from tissue absorption and scattering coefficients, tissue conductivity and convection by blood, as described above. Clearly, certain parameters may be found to be less important or redundant in practical clinical work. Thus, the present patent disclosure may be applied with all available measurement modalities, or by just using a limited number of them. However, to distinguish the present invention over prior art with regard to optical measurements, temperature and temporal dynamics will always be assessed in addition to light flow measurements in this combined PDT/thermal treatment modality.

It should be noted, that initially PDT action occurs also in the tissue areas close to the fibers, but the action would soon largely stop due to thermal disruption of the PDT action. This is no harm, since the tissue heating would independently kill the cells.

Because of tissue modification (change in tissue optical parameters) due to tissue coagulation, certain initial properties pertinent to PDT should be measured before the tissue is subject to excessive heat-generating laser irradiation. This pertains particularly to sensitizer concentration, and also to light flux and degree of oxygenation.

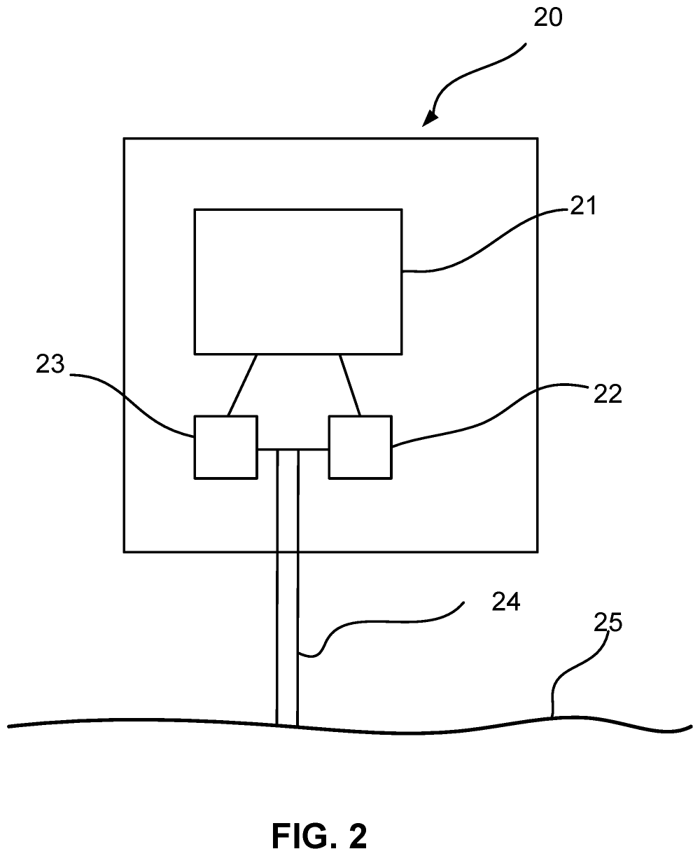

Because of changes in the optical and thermal properties of tissue during a full treatment session, periods of administration of treatment light should be interrupted by periods of assessing tissue characterization parameters as discussed above. In the calculation of treatment progression (calling for interactive changes in light administration), thermal dosimetry and PDT dosimetry (for the peripheral tissue areas) can largely be handled separately, while corresponding data at times might be of mutual value. The treatment is considered concluded when threshold dose values for the thermal and PDT modalities have been reached in all relevant tissue areas, subject to assuring that organs at risk remain well under the damage limits. This assessment is made in the combined PDT/Thermal dosimetry program. As in earlier IPDT approaches, relevant volumes and boundaries are determined, e.g., by in-situ ultrasound, with prior data from, e.g., x-ray CT or magnetic resonance imaging (MRI) possibly integrated into the medical imagery which forms the base for dose planning. FIG. 2 is illustrating a schematic example of a system 20 for treatment of malignant tumors. The system may include at least two optical members 24, such as a plurality of optical members 24, with a distal portion configured to be interstitially inserted into tissue 25, as illustrated in FIG. 1. The optical members 24 may be optical waveguides or optical fibres. The at least two optical members 24 are configured for emitting light to the tissue 25 and for collecting light from the tissue 25. A distal end (not shown) of the optical member 24 may be used to collect back scattered light from an area to be measure or diagnosed, such as a tissue site, or a tumour.

The system 20 may further include a light source 22 for emitting light to the tissue 25 using at least one of the at least two optical members 24. The system may further include a detector 23 for detecting collected light from at least one of the at least two optical members 24.

The detector 23 may be, e.g., photodiodes, photomultiplier tubes, avalanche photodiodes, charge-coupled devices (CCD), or CMOS light sensitive devices.

In an example, light sources 22 may be a lamp, photodiodes, e.g., light emitting diodes (LED) or laser diodes. The light source may have one or more filters for filtering the wavelength of the light emitted.

The light sources 22 may be diagnostic light sources having a wavelength corresponding to the absorption of one or more chromophores in tissue, such as deoxy-hemoglobin and/or oxy-hemoglobin. The light sources 22 may also be the same as the light sources used for treatment of the tissue site, such as treatment of a tumour.

When performing both diagnostic and treatment, they may be carried our sequentially. For example, a period of treatment may be followed by a period of diagnostic. In some example, the treatment may be carried out simultaneously as the diagnostic.

The system may further include a control unit 21 configured for controlling the system so that light is transmitted to the tissue 25 from at least one optical members 24 and light is detected from the tissue 25 by at least one optical member 24 collecting light. This may yield a data set of measured values for pairs of emitting and collecting optical members 24.

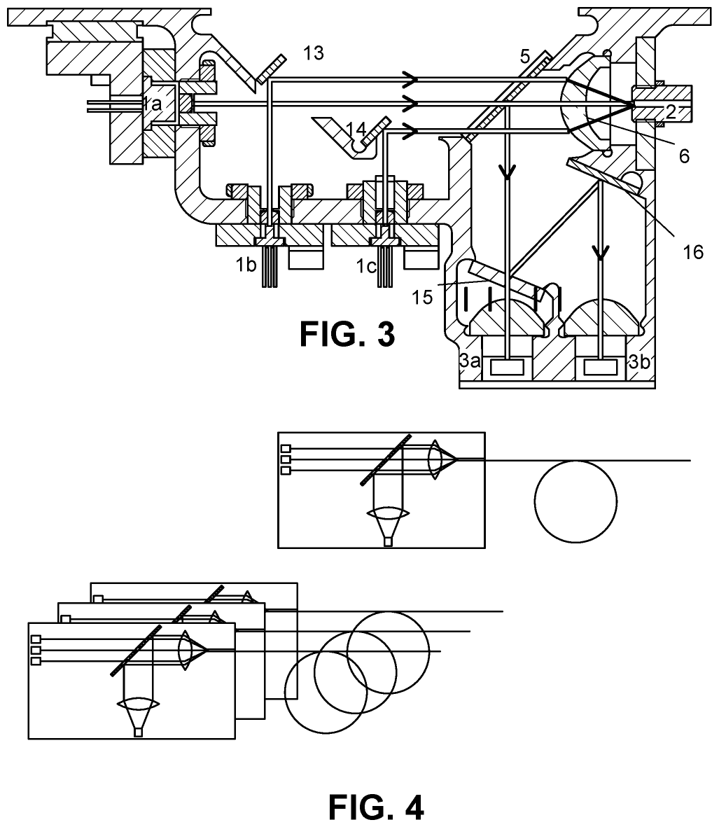

For example, a transmission member of the optical members is sequentially selected between the plurality of optical members 24. This may be done by sequentially switching on and off the light emitting part of a plurality of modules. An example of a module is illustrated in FIG. 3 and a plurality of modules are illustrated in FIG. 4.

In some example may the system 20 include a plurality of optical members 24. In some examples one single fibre at a time may emit light, and all other fibres collecting. Alternatively, in some example other measurement regimens may be possible, such as using a sub-set of all fibres that emit, or a sub-set of all fibres that collect, or combinations of these.

In some examples of the system, may the measured and/or determined optical properties of the tissue be used for calculating a light dose for photodynamic therapy and/or laser thermal treatment.

In some examples are the plurality of optical members configured to be arranged on the tissue site so that a spatially resolved measurement may be performed. The optical properties may then be obtained from the measurements by solving the transport equation of radiative transfer.

In some example of the system, may the optical members be optical fibres, or optical fibres with diffusers. The optical member may be configured to be interstitially arranged in tissue to allow treatment and/or diagnosis of a deep laying tissue site. In one example, this may be done using needles, syringes and/or a catheter.

Alternatively, in some examples, the optical members are configured to transfer light to and from a surface of a tissue site, such as a skin surface or a surface in a body cavity.

The control unit 21 may also be configured to control a power of the light source 22 so that a combination of a laser-light thermal therapy and a light-activated photodynamic therapy is conducted wherein regions close to emitting fibers being eradicated by thermal effects, while regions further away being treated with the light-activated photodynamic therapy and tumor-cell selectivity.

The control unit, data processing device or a computing device may be implemented by special-purpose software (or firmware) run on one or more general-purpose or special-purpose computing devices. In this context, it is to be understood that each “element” or “means” of such a computing device refers to a conceptual equivalent of a method step; there is not always a one-to-one correspondence between elements/means and particular pieces of hardware or software routines. One piece of hardware sometimes comprises different means/elements. For example, a processing unit serves as one element/means when executing one instruction, but serves as another element/means when executing another instruction. In addition, one element/means may be implemented by one instruction in some cases, but by a plurality of instructions in some other cases. Such a software controlled computing device may include one or more processing units, e.g. a CPU (“Central Processing Unit”), a DSP (“Digital Signal Processor”), an ASIC (“Application-Specific Integrated Circuit”), discrete analog and/or digital components, or some other programmable logical device, such as an FPGA (“Field Programmable Gate Array”). The control unit, data processing device or computing device may further include a system memory and a system bus that couples various system components including the system memory to the processing unit. The system bus may be any of several types of bus structures including a memory bus or memory controller, a peripheral bus, and a local bus using any of a variety of bus architectures. The system memory may include computer storage media in the form of volatile and/or non-volatile memory such as read only memory (ROM), random access memory (RAN) and flash memory. The special-purpose software may be stored in the system memory, or on other removable/non-removable volatile/non-volatile computer storage media which is included in or accessible to the computing device, such as magnetic media, optical media, flash memory cards, digital tape, solid state RAM, solid state ROM, etc. The data processing device may include one or more communication interfaces, such as a serial interface, a parallel interface, a USB interface, a wireless interface, a network adapter, etc., as well as one or more data acquisition devices, such as an A/D converter. The special-purpose software may be provided to the control unit or data processing device on any suitable computer-readable medium, including a record medium and a read-only memory.

Any method or determinations or calculations described herein may be implemented on a control unit, a data processing device or a computing device. The computer implementation may be done as a computer program or a software to be executed on a control unit, a data processing device or a computing device.

Any methods or determinations or calculations described herein may be performed by a control unit, a data processing device or a computing device. The control unit, data processing device or computing device may be connected to the optical detection system or be a standalone unit.

In FIG. 3, light beams from each light source 1a, 1b, 1c may be separated spatially using reflective components 13, 14, such as a mirror, or by positioning two mirror next to each other with a gap in between, wherein the gap will be a slit. In this example, one of the light sources 1a is arranged along an optical axis which the other two are arranged at an angle, such as perpendicular, to the optical axis. By arranging the reflective members 13, 14 at different distances from the optical axis, the light may be reflected from the other light sources 1b, 1c to obtain three parallel light paths and/or beam paths.

Each of the parallel light paths and/or beam paths may pass through an aperture, in a reflective member 5, as described in WO 2021/240023 A1 which is incorporated by reference. The light may then be focused into the optical member 2 by a focusing component 6. Most of the returning light from the optical member 2 may be reflected by the reflective member 5, to the beam splitter 15, which reflects light with the wavelength of light source 1a (the shortest wavelength), while light with longer wavelength than that of light source 1a may be transmitted and may be detected by the detector 3a. The light that is reflected by the beam splitter 15 may be detected by detector 3b or may be reflected by a further reflective element 16, such as a mirror, which will direct the light towards the detector 3b. With this arrangement, light at the wavelength of light source 1a may be detected simultaneously with light from either light source 1b or 1c. Alternatively, the light from light source 1a may induce fluorescence in the tissue at wavelengths longer than the wavelength of light source 1a, for example fluorescence by a photosensitizer as well as heating the tissue close to the distal end. In such a case, the fluorescence light may be detected by detector 3a, separated from the light from light source 1a which will be detected by detector 3b. The light sources 1b and 1c may be switched on sequentially after light source 1a and the detected light may be detected with detector 3a.

In some examples, a plurality of modules are combined into a single complete system, so that the modules may interact in the manner described in the patent EP 1 443 855 A1. FIG. 4 illustrates a system based on a plurality of modules such as illustrated in FIG. 3, where a plurality of optical fibres may be connected to said system. The system may include at least two optical members configured to be inserted into tissue, such as interstitially inserted. The at least two optical members may be configured for emitting and/or collecting light.

The present invention has been described above with reference to specific examples. However, other examples than the above described are equally possible within the scope of the disclosure. Different method steps than those described above, performing the method by hardware or software, may be provided within the scope of the invention. The different features and steps of the invention may be combined in other combinations than those described. The scope of the disclosure is only limited by the appended patent claims.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases.

1. A system for treatment of malignant tumors, wherein the system comprises:
at least two optical members with a distal portion configured to be interstitially inserted into tissue, the at least two optical members are configured for emitting and collecting light;
a light source and a detector connected to the optical members at a proximal end; and
a control unit configured for controlling the at least two optical members so that light is transmitted to the tissue from at least one optical member and light is detected from the tissue by at least one optical member collecting light;
wherein the control unit is further configured to control a power of the light source so that a combination of a laser-light thermal therapy and a light-activated photodynamic therapy is conducted wherein regions close to emitting optical members being eradicated by thermal effects, while regions further away being treated with the light-activated photodynamic therapy and tumor-cell selectivity.

2. The system according to claim 1, light source is emitting a single laser wavelength used for both the laser-light thermal therapy and the light-activated photodynamic therapy.

3. The system according to claim 1, wherein the light source is adapted to emitting a wavelength matching requirements for a sensitizer being Protoporphyrin IX generated from the precursor δ-aminolevulinic acid (ALA), or an ALA-related formulation, used for the light-activated photodynamic therapy.

4. The system according to claim 1, wherein the system is configured for diagnostics which is based on at least one of the parameters: light flow between optical members, oxygenation in the tissue, concentration of sensitizer in the tissue, blood flow through the tissue, temperature at the individual optical member tips, and/or temperature dynamics.

5. The system of claim 4, wherein the parameters are derived from light detected by the collecting optical members.

6. The system according to claim 1, wherein the control unit is configured to use measured optical data from the collecting optical members as input for treatment planning and interactive follow-up handling aspects related to the laser-light thermal therapy separately, and aspects related to the light-activated photodynamic therapy separately, using relevant models for tissue heating, heat flow, and PDT action.

7. The system according to claim 4, where at least the light flow between optical members, the temperatures at the optical member tips, and the temperature dynamics are measured as input in the combined PDT and thermal dosimetry program, where the respective threshold doses have been delivered, and organs at risk have doses well under thresholds.

8. The system of claim 7, wherein at least the light flow between optical members, the temperatures at the optical member tips, and the temperature dynamics are expressed in time constants, or other time-dependent parameters.

9. The system according to claim 1, wherein the control unit is configured to extract beat signals in the kHz frequency range caused by Doppler shifts in light interaction with flowing blood cells.

10. The system according to claim 1, wherein each emitting optical member is connected to an individual light source having an output power in the range 0.1-5 W.

11. The system according to claim 1, wherein the light source outputs the light at a wavelength in the range of 625-640 nm.

12. A method of treatment of malignant tumors, wherein the method comprises:
inserting at least two optical members with a distal portion configured to be interstitially inserted into tissue, the at least two optical members are configured for emitting and collecting light and are connected to a light source and a detector at a proximal end;
controlling the at least two optical members so that light is transmitted to the tissue from at least one optical member and light is detected from the tissue by at least one optical member collecting light; and
controlling a power of the light source so that a combination of a laser-light thermal therapy and a light-activated photodynamic therapy is conducted wherein regions close to emitting optical members being eradicated by thermal effects, while regions further away being treated with the light-activated photodynamic therapy and tumor-cell selectivity.

13. A computer readable media having stored thereon a computer program to be executed on a control unit of the system according to claim 1, wherein the computer program comprises machine readable instructions causing the system to perform the steps of:
controlling at least two optical members so that light is transmitted to the tissue from at least one optical member and light is detected from the tissue by at least one optical member collecting light; and
controlling a power of the light source so that a combination of a laser-light thermal therapy and a light-activated photodynamic therapy is conducted wherein regions close to emitting optical members being eradicated by thermal effects, while regions further away being treated with the light-activated photodynamic therapy and tumor-cell selectivity.
